(19) United States
(12) Patent Application Publication
(10) Pub. No.: US 2024/0325573 A1
(43) Pub. Date: Oct. 3, 2024

(54) X-RAY DEVELOPABLE MOLECULE, EMBOLISM MICROSPHERE AND PREPARATION METHODS THEREFOR

(71) Applicant: Shanghai Huihe Healthcare Technology Co., Ltd., Shanghai (CN)

(72) Inventors: Xuefei ZHANG, Shanghai (CN); Chenyi LEI, Shanghai (CN); Bingqing WANG, Shanghai (CN)

(21) Appl. No.: 18/740,530

(22) Filed: Jun. 12, 2024

Related U.S. Application Data
(63) Continuation of application No. PCT/CN2022/135728, filed on Dec. 1, 2022.

(30) Foreign Application Priority Data
Dec. 30, 2021 (CN) 202111648826.5

(51) Int. Cl. A61K 49/04; A61L 24/00; A61L 24/06; A61L 24/08

(52) U.S. Cl. CPC A61K 49/0438; A61L 24/001; A61L 24/06; A61L 24/08; A61L 2300/202

(57) ABSTRACT

An X-ray developable molecule, an embolism microsphere and preparation methods therefor, in the technical field of medical materials, are disclosed. The methods include preparing the X-ray developable molecule by reacting a compound with an amino group and an aldehyde, hemiacetal or acetal group with an iodobenzoic acid derivative, the X-ray developable molecule having an amide group; and/or reacting the X-ray developable molecule with a polyhydroxy polymer microsphere to prepare the X-ray developable embolism microsphere. The microsphere has X-ray developing and drug loading properties, and the methods of preparation and use are simple, so that a doctor can directly observe a body part containing the embolism microsphere using X-ray fluoroscopy. Intraoperative use is facilitated, the embolism extent is easy to determine, and complications in the intravascular treatment process are effectively avoided.

### CROSS-REFERENCE TO RELATED APPLICATIONS

This application is a continuation of International Pat. Appl. No. PCT/CN2022/135728, filed on Dec. 1, 2022, which claims priority to Chinese Pat. Appl. No. 202111648826.5, filed on Dec. 30, 2021, the contents of each of which are incorporated by reference herein in their entireties.

### TECHNICAL FIELD

The present invention relates to the technical field of medical materials, in particular to an X-ray developable molecule, an embolism microsphere (e.g., containing the X-ray developable molecule), and a methods of preparing the same.

### BACKGROUND

In recent years, interventional embolization therapy has played an increasingly important role in clinical medicine, especially in the treatment of vascular-rich tumors such as those associated with liver cancer, and has become the preferred alternative for treating tumors that cannot be surgically removed. The embolism microsphere is one of the most common embolism carriers at present and has received increasing attention due to its high targeting to specific tissues and organs, good ability to form embolisms, and its compatibility with chemotherapeutic drugs and slow-release drugs. At present, commercially available microspheres (such as DC Bead and CalliSphere) generally have a uniform size, smooth surfaces, good flexibility and elasticity, and good hydrophilicity and ability to form a suspension. They are easy to guide with blood flow and can block the full cross-section of blood vessels, slowly release drugs at the lesion site, maintain a local effective drug concentration for a long time, and have a significant cytotoxic effect on tumor cells. However, these microspheres do not have visibility using (e.g., opacity to) X-rays, and the embolization effect can only be observed by angiography when the microspheres are injected into the target blood vessel.

CN 108686259B introduces a drug-loading microsphere capable of developing under X-rays for intravascular embolization, and the microsphere consists of polyvinyl alcohol and polyacrylic acid, and contains a barium precipitate inside. Since barium is a high-density metallic element, microspheres containing barium precipitate have the characteristic of being opaque to X-rays. However, the barium precipitate is physically embedded inside the microsphere, which may seep out freely in the blood vessels, affecting the safety of the embolic agent.

CN 105517580A introduces a preparation method of an imagable embolism microsphere by activating pre-formed hydrogel beads by nucleophilic attack of iodized compounds, and then connecting the iodized compounds to the microsphere. However, the steps of this method are cumbersome, and the preparation process of the microsphere requires a long reaction time (more than 24 hours) and relatively harsh reaction conditions.

CN 111821503A describes a radiopaque embolism microsphere that is connected to an iodine-substituted alkyl (sulfonyl) chloride derivative and exhibits X-ray developing effects. However, this method requires the use of highly toxic organic solvents such as NMP and THF in the synthesis of the microspheres, and the reaction system requires strict water removal under harsh conditions.

### SUMMARY

The purpose of the present invention is to provide an X-ray developable molecule, an embolism microsphere (e.g., containing the X-ray developable molecule), and methods of preparing the same. The microsphere has X-ray developing properties (e.g., radiopacity) and drug loading properties, and the preparation method is relatively simple, so that a doctor can directly observe the body part(s) containing the embolism microspheres by X-ray fluoroscopy and other X-ray imaging techniques. The present microspheres facilitate intraoperative detection and/or imaging, determination of the degree or extent of embolism formation, and avoidance of various complications in intravascular treatment processes.

The technical solution(s) provided by the present invention include the following:

The present invention provides an X-ray developable molecule, having the Formula I:

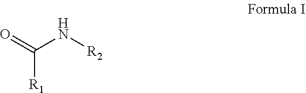

wherein R₁ is a phenyl group substituted with 1 to 4 iodine atoms, 0 or 1 —NH₂ groups, and 0 or 1 —C(═O)—NH—R₂ groups, and R₂ is a structure containing an aldehyde, a hemiacetal, or an acetal. For example, R₁ may be an iodobenzene derivative selected from the following:

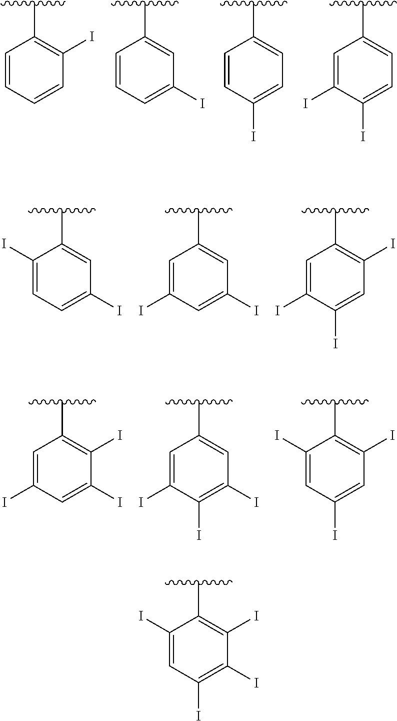

and R₂ may be selected from the group consisting of

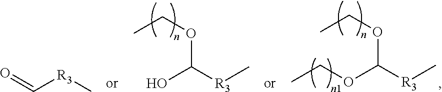

where R₃ is an aryl group or a saturated or unsaturated C₁-C₆ alkylene group, and n and n₁=0-3.

In further embodiments of the present invention, the X-ray developable molecule may include one or more of the following compounds:

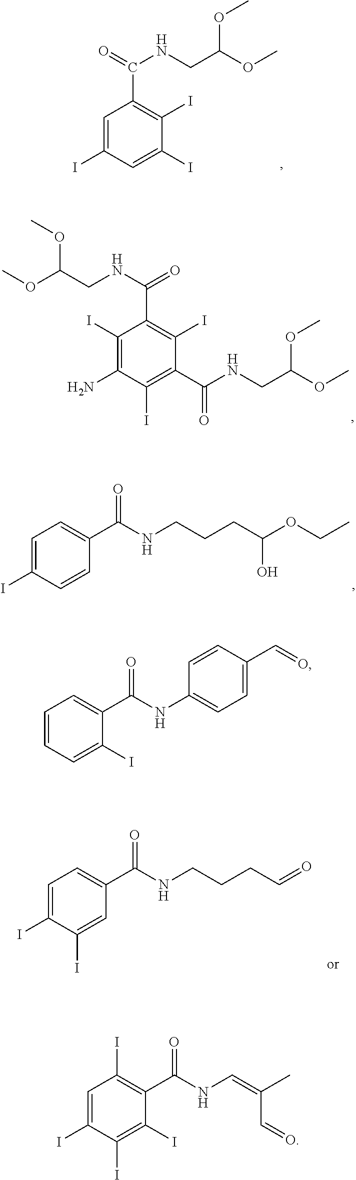

The present invention further includes a method of preparing the X-ray developable molecule, comprising reacting a compound containing an amino group and the aldehyde, hemiacetal or acetal group with an iodobenzoic acid derivative (to obtain the X-ray developable molecule).

In further embodiments of the present invention, the compound with an amino group and an aldehyde, hemiacetal or acetal group may be:

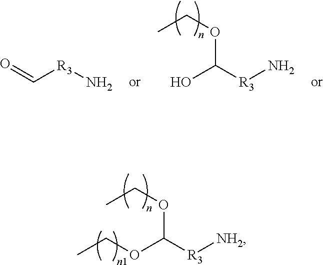

where R₃ is an aryl group or a saturated or unsaturated C₁-C₆ alkylene group, n=0-3 and n₁=0-3. Preferably, R₃ is a C₁-C₂ alkylene group, where n=0 and n₁=0.

In further embodiments of the present invention, the iodobenzoic acid derivative contains both the R₁ group and a hydroxyl, a carboxyl, an acyl chloride or an acyl bromide group, preferably selected from one or more of:

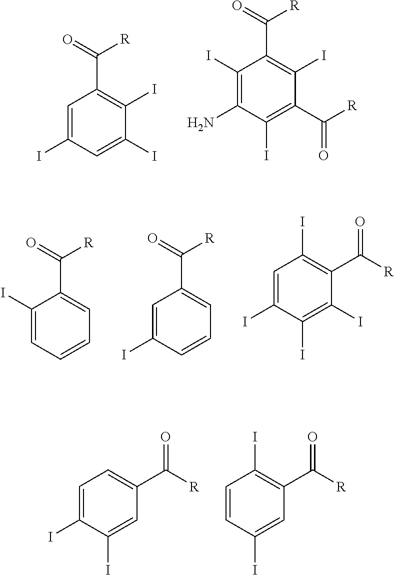

where R═Br, Cl or OH.

In further embodiments of the present invention, reacting the compound with the iodobenzoic acid derivative may comprise adding the compound, the iodobenzoic acid derivative and an alkali to an organic solvent, reacting the compound, the iodobenzoic acid derivative and the alkali at a temperature of 0-40° C. for a time of 0.5-48 hours, and removing the solvent to obtain the X-ray developable molecule. The compound, the iodobenzoic acid derivative and the alkali may be fed into the organic solvent at a temperature of from −10° C. to 25° C. under an inert gas atmosphere, and the compound, the iodobenzoic acid derivative and the alkali may also be reacted under the inert gas atmosphere. After the reaction, the reaction mixture (e.g., containing the X-ray developable molecule, the alkali and the organic solvent) may be washed (e.g., with deionized water, saturated salt [NaCl] water, a dilute mineral acid, etc.) and/or the X-ray developable molecule may be extracted (e.g., from the organic solvent and/or the aqueous washing agent, or after removing the organic solvent). The compound with the amino group and the aldehyde, hemiacetal or acetal group may be present in the organic solvent in a concentration (e.g., an amount-of-substance concentration) of 0.01-3 mol/L, preferably 0.1-1 mol/L, and the iodobenzoic acid derivative may be present in the organic solvent in an independent concentration (e.g., an amount-of-substance concentration) of 0.01-3 mol/L, preferably 0.1-1 mol/L.

In further embodiments of the present invention, the reaction may be conducted at a temperature of 20-30° C. for a time of 2-24 hours. The temperature at which the compound, the iodobenzene derivative and the alkali are fed into the organic solvent may be from −5° C. to 5° C., and such feeding may be conducted under the inert gas atmosphere.

In further embodiments of the present invention, the alkali comprises an inorganic alkali or an organic alkali. For example, the alkali may be selected from the group consisting of sodium hydroxide solution, potassium hydroxide solution, diethylamine, ethylenediamine, triethylamine, ammonia, pyridine, sodium methoxide, and sodium hydride.

In further embodiments of the present invention, the alkali may be present (e.g., in the organic solvent) in a concentration (e.g., an amount-of-substance concentration) of 0.01-2 mol/L, preferably 0.1-1 mol/L.

In further embodiments of the present invention, the organic solvent may comprise or be selected from the group consisting of dimethyl sulfoxide, tetrahydrofuran, dichloromethane, chloroform, methanol, acetone, acetonitrile, ether, N-methylpyrrolidone, N, N-dimethylformamide.

The present invention further sets forth an X-ray developable embolism microsphere containing the above X-ray developable molecule and a polyhydroxy polymer microsphere, where the X-ray developable molecule is bound or coupled to the polyhydroxy polymer microsphere through an acetal group or link. The microsphere may have a particle size of 1-1,500 microns.

In further embodiments of the present invention, to improve the drug loading properties of the microsphere, the microsphere may comprise a crosslinked copolymer of the polyhydroxy polymer with a water-soluble compound containing one or more unsaturated bonds and one or more aldehyde or acetal groups. The copolymer may be cross-linked by a crosslinking agent comprising a second water-soluble compound containing one or more anionic functional groups and one or more unsaturated bonds, which may be selected from the group consisting of carboxylic acid compounds and carboxylate salts thereof having or generating one or more carboxylate groups and having one or more unsaturated bonds, and sulfonic acid compounds and sulfonate compounds having or generating one or more sulfonate groups and having one or more unsaturated bonds. In other embodiments, the polyhydroxy polymer microsphere comprises a polyhydroxy polymer crosslinked with a water-miscible compound containing two or more aldehyde or acetal groups. The water-miscible compound may comprise a C₃-C₆ α,ω-alkane dialdehyde or a C₃-C₆ α,α,ω,ω-tetra(C₁-C₄ alkoxy)alkane, such as glutaraldehyde, 1,1,5,5-tetramethoxypentane, succinaldehyde, 1,1,4,4-tetra(t-butoxy)butane, malonaldehyde, and 1,1,3,3-tetraethoxypropane.

In further embodiments of the present invention, the carboxylic acid compounds and the carboxylate salts thereof may be selected from the group consisting of acrylic acid, methacrylic acid, sodium acrylate, and sodium methacrylate. The sulfonic acid compounds and the sulfonate compounds may be selected from the group consisting of 2-acrylamido-2-methylpropanesulfonic acid, 2-acrylamido-2-methylpropanesulfonic acid sodium salt, 3-sulfopropyl acrylate potassium salt, and 3-sulfopropyl methacrylate potassium salt.

In further embodiments of the present invention, the X-ray developable embolism microsphere contains iodine in an amount greater than or equal to 30 mg/g of the microspheres (e.g., of the microspheres in a dry state), preferably greater than or equal to 100 mg/g of the microspheres. The iodine content of the embolism microspheres is generally less than or equal to 500 mg/g.

The present invention also concerns a method of preparing the X-ray developable embolism microsphere, comprising coupling or connecting the X-ray developable molecule with the polyhydroxy polymer microsphere. More specifically, the method comprises adding dissolving the polyhydroxy polymer microsphere and the X-ray developable molecule in a solvent, adding an acid (e.g., to the polyhydroxy polymer microsphere and the X-ray developable molecule in the solvent), reacting the polyhydroxy polymer microsphere and the X-ray developable molecule (e.g., in the presence of the acid), and removing the solvent. After the reaction, or after removing the solvent, the method may further comprise washing a product of the reaction (e.g., the X-ray developable embolism microsphere) with a washing agent (e.g., deionized water, saturated salt water, a dilute and/or mild aqueous base such as 1-6 M aqueous sodium or potassium bicarbonate, etc.).

In further embodiments of the present invention, the polyhydroxy polymer comprises a polymer or polysaccharide macromolecule with 1,2-glycol or 1,3-glycol groups or linkages (e.g., having 1,2 and/or 1,3 glycosidic links between adjacent saccharide units).

In further embodiments of the present invention, the polyhydroxy polymer may be selected from the group consisting of polyvinyl alcohol, chitosan, hyaluronates, alginates, amylose, cellulose and modified cellulose.

In further embodiments of the present invention, the acid is an organic or inorganic acid selected from the group consisting of hydrochloric acid, sulfuric acid, nitric acid, methanesulfonic acid, acetic acid (e.g., glacial acetic acid), citric acid, benzoic acid, and perchloric acid.

In further embodiments of the present invention, the solvent may comprise a polar solvent. For example, the solvent may be selected from the group consisting of dimethyl sulfoxide, water, acetone, acetonitrile, and N-methylpyrrolidone.

In further embodiments of the present invention, the mass fraction of the polyhydroxy polymer microsphere in the solvent is 1%-30%, preferably 5%-15%; the concentration (e.g., an amount-of-substance concentration) of the X-ray developable molecule in the solvent is 0.01-2 mol/L, preferably 0.05-0.5 mol/L; and the concentration (e.g., the amount-of-substance concentration) of the acid in the solvent is 0.05-10 mol/L, preferably 0.5-5 mol/L.

In further embodiments of the present invention, the reaction may be conducted at a temperature of from room temperature to 120° C., preferably from room temperature to 80° C., and for a length of time of from 15 minutes to 48 hours, preferably from 30 minutes to 24 hours.

In order to improve the drug loading properties of the microsphere, the present invention further concerns a method of preparing the polyhydroxy polymer microsphere, comprising:

S1. Dissolving the polyhydroxy polymer in water, then adding the water-soluble compound containing one or more unsaturated bonds and one or more aldehyde or acetal groups, and an inorganic acid; reacting the polyhydroxy polymer with the water-soluble molecule (e.g., in the presence of the inorganic acid) to form a solution containing a microsphere intermediate; after the reaction is completed, adjusting the pH of the reaction mixture (e.g., containing the microsphere intermediate) to 7-9, concentrating the solution, and obtaining the microsphere intermediate. The reaction time may affect the yield, so the reaction is usually conducted at a temperature of 10-35° C. for 3-8 hours, although this aspect of the invention is not so limited. To achieve a high yield, a reaction time at the longer end of the range (e.g., 6-8 hours, and in some cases, up to 12-24 hours) can be chosen. The yield may also be improved by concentrating the solution (e.g., the reaction mixture) to a viscosity greater than or equal to 1,500 cps, preferably around 1,800 cps.

S2. Dissolving the microsphere intermediate prepared in step S1, a crosslinking agent (e.g., containing one or more anionic functional groups, such as a carboxylate or sulfonate group, and one or more unsaturated bonds), and an initiator in water, adding a solvent (e.g., a water-insoluble organic solvent), a surfactant (e.g., to form an inverse suspension polymerization system), and an organic alkali to the water (e.g., under an inert gas atmosphere), reacting the microsphere intermediate, the crosslinking agent, and the initiator under the inert gas atmosphere, and after the reaction is completed, filtering and washing the resulting reaction mixture to obtain the polyhydroxy polymer microspheres. The reaction temperature can be 55-65° C., and the reaction time can be 2-6 hours.

The microspheres prepared by the above method are bound or connected to the X-ray developable molecule, so that the microspheres have excellent developing properties (e.g., visibility or contrast in X-ray imaging) and improved the drug loading properties. The drugs that can be loaded in and/or on the microspheres may comprise drugs with a positive charge in an aqueous solution, which can be selected from adriamycin, epirubicin, pirarubicin, 5-fluorouracil, capecitabine, 6-mercaptopurine, gemcitabine, irinotecan, bleomycin, oxaliplatin, sorafenib, sunitinib, raltitrexed, endostar, topotecan, and mitomycin, although the invention is not limited to these examples.

In further embodiments of the present invention, the polyhydroxy polymer, the water-soluble compound containing one or more unsaturated bonds and aldehyde or acetal groups, and the inorganic acid may be present in step S1 in a mass ratio of 1:(0.01-0.5):(0.05-5).

In further embodiments of the present invention, the microsphere intermediate, the crosslinking agent, the initiator, the water, the solvent, the surfactant and the organic alkali may be present in step S2 in a mass ratio of 1:(0.001-0.2):(0.0001-0.05):(0.1-3):(4-50):(0.001-0.1):(0.0001-0.05).

In further embodiments of the present invention, the initiator may be selected from the group consisting of potassium persulfate, ammonium persulfate, and sodium persulfate; and the crosslinking agent may be selected from the group consisting of carboxylic acid compounds and carboxylate salts thereof having or generating one or more carboxylate groups and having one or more unsaturated bonds, and sulfonic acid compounds and sulfonate compounds having or generating one or more sulfonate groups and having one or more unsaturated bonds. The carboxylic acid compounds and carboxylate salts thereof may be selected from the group consisting of acrylic acid, methacrylic acid, sodium acrylate, and sodium methacrylate. The sulfonic acid compounds and sulfonate compounds may be selected from the group consisting of 2-acrylamido-2-methylpropanesulfonic acid, 2-acrylamido-2-methylpropanesulfonic acid sodium salt, 3-sulfopropyl acrylate potassium salt, and 3-sulfopropyl methacrylate potassium salt. The water-soluble compound containing one or more unsaturated bonds and one or more aldehyde or acetal groups may be selected from the group consisting of N-(2,2-dimethoxyethyl)-2-acrylamide, N-acrylamidoacetaldehyde diethyl acetal, 4-acrylamide butyraldehyde dimethyl acetal, N-acrylamide acetal, and 4-acrylamide phenyl acetal. The inorganic acid in step S1 may be concentrated hydrochloric acid or concentrated sulfuric acid (e.g., as a catalyst). The solvent in step S2 may be selected from the group consisting of butyl acetate, ethyl acetate, liquid paraffin, castor oil, soybean oil, n-heptane or cyclohexane; the surfactant may be selected from the group consisting of cellulose acetate butyrate, cellulose acetate, Span 20, Span 80, Tween 20, and Tween 80; and the organic alkali in step S2 may be selected from the group consisting of tetramethyl ethylenediamine, ethylenediamine, triethylamine, and N,N-dimethylaniline (e.g., as a catalyst).

The present invention has one or more of the following beneficial effects:

The present invention provides an X-ray developable embolism microsphere and a preparation method therefor. The microsphere has one or more X-ray developing properties (e.g., radiopacity) and one or more drug loading properties, and the preparation method is relatively simple. A doctor can directly observe the body part(s) containing the embolism microspheres using X-ray fluoroscopy or another X-ray imaging technique. Intraoperative diagnoses and processes are facilitated, the degree or extent of the embolism is easy to determine, and various complications in intravascular treatment processes are effectively avoided.

The drug-loading microsphere of the present invention has an X-ray developing or imaging function and beneficial drug loading properties

The method of preparing the X-ray developing, drug-loading microsphere of the present invention is relatively simple and safe to human health (low temperature, short reaction time, less use of toxic or potentially solvents, and a high yield of radiopaque species connecting to the microspheres).

### BRIEF DESCRIPTION OF THE DRAWINGS

In order to illustrate the technical solutions in the embodiments of the present invention (or the problems in the prior art) more clearly, the drawings for the embodiments or prior art will be introduced briefly. Obviously, the drawings described herein represent just some of the embodiments of the present invention. For the person of ordinary skill in the art, they can also derive or conceive other drawings based on these drawings, without creative effort.

FIG. 1 is a microscope image of an X-ray developable embolism microspheres prepared according to Embodiment 1 of the present specification;

FIG. 2 shows X-ray images of the X-ray developable polyvinyl alcohol embolism microspheres prepared according to Embodiment 1 of the present specification and non-developable polyvinyl alcohol microspheres using digital subtraction angiography (DSA) technology;

FIG. 3 is a microscope image of the microspheres prepared according to Comparative Example 1 of the present specification;

FIG. 4 shows the ¹H NMR spectroscopy of N-(2,2-dimethoxyethyl)-2,3,5-triiodobenzamide prepared according to Embodiment 1 of the present specification;

FIG. 5 shows the infrared spectrum of the X-ray developable embolism microspheres prepared according to Embodiment 1 of the present specification;

FIG. 6 shows the ¹H NMR spectroscopy of 5-amino-1,3-bis(2,2-dimethoxyethyl)-2,4,6-triiodoisophthalamide prepared according to Embodiment 2 of the present specification;

FIG. 7 shows the infrared spectrum of the X-ray developable embolism microspheres prepared according to Embodiment 2 of the present specification; and

FIG. 8 shows an X-ray image of the X-ray developable polyvinyl alcohol embolism microspheres prepared according to Embodiment 7 of the present specification.

### DETAILED DESCRIPTION OF THE PREFERRED EMBODIMENTS

The technical solutions in the embodiments of the present invention will be described clearly and completely in the following paragraphs. Obviously, the described embodiments are only a portion of the embodiments of the present invention, not all of the embodiments of the present invention. All other embodiments envisioned or conceived by those skilled in the art without inventive step based on the embodiments of the present invention are considered within the scope of the present invention.

Dry microspheres refer to those obtained by the complete or substantially complete volatilization or removal of water or other solvents from the microspheres.

In the present invention, unless otherwise specified, the concentration of concentrated hydrochloric acid is 37.0-37.5 wt %, and the concentration of concentrated sulfuric acid is 98.0-98.5 wt %.

### Preparation Example 1—Preparation of Polyvinyl Alcohol Microspheres

S1. Polyvinyl alcohol (10 g) with an average molecular weight of 67,000 g/mol is added to 100 mL of purified water and dissolved completely at 90° C. N-(2,2-dimethoxyethyl)-2-acrylamide (5 g) and concentrated hydrochloric acid (42 mL) are added to the aqueous polyvinyl alcohol and reacted at 30° C. for 8 hours. After the reaction is completed (i.e., the 8 hours passes), the pH of the reaction mixture is adjusted to 7 with aqueous sodium hydroxide. Finally, the pH-adjusted reaction mixture is concentrated until its viscosity is 2,200 cps, to obtain a microsphere intermediate.

S2. The above microsphere intermediate (15 g), 2-acrylamido-2-methylpropanesulfonic acid sodium salt (3 g), and potassium persulfate (0.75 g) are completely dissolved in 5 mL of deionized water, then butyl acetate (219 mL) and cellulose acetate butyrate (1.5 g) are added. Finally, tetramethyl ethylenediamine (0.75 g) are added to the mixture under a nitrogen atmosphere and reacted at 65° C. for 6 hours. After the reaction is completed (i.e., the 6 hours passes), the reaction mixture is filtered and washed with ethyl acetate, acetone, and deionized water (e.g., in succession) to obtain crosslinked polyvinyl alcohol microspheres.

### Preparation Example 2—Preparation of Polyvinyl Alcohol Microspheres

S1. Polyvinyl alcohol (60 g) having an average molecular weight of 62,000 g/mol is added to 400 mL of purified water and dissolved completely at 90° C. N-acrylamidoacetaldehyde diethyl acetal (0.6 g) and concentrated sulfuric acid (1.7 mL) are added to the aqueous polyvinyl alcohol and reacted at 10° C. for 4 hours. After the reaction is completed (i.e., the 4 hours passes), the pH of the reaction mixture is adjusted to 9 with aqueous sodium hydroxide. Finally, the pH-adjusted reaction mixture is concentrated until its viscosity is 1,800 cps, to obtain a microsphere intermediate.

S2. The above microsphere intermediate (75 g), 2-acrylamido-2-methylpropanesulfonic acid sodium salt (0.075 g), and potassium persulfate (0.0075 g) are completely dissolved in 20 mL of deionized water, then ethyl acetate (8.4 mL) and cellulose acetate butyrate (0.075 g) are added, and finally ethylenediamine (0.009 mL) is added under a nitrogen atmosphere. The mixture is reacted at 55° C. for 2 hours. After the reaction is completed (i.e., the 2 hours passes), the reaction mixture is filtered and washed with ethyl acetate, acetone, and deionized water (e.g., in succession) to obtain the crosslinked polyvinyl alcohol microspheres.

### Preparation Example 3—Preparation of Sodium Alginate Microspheres

S1. Sodium alginate (10 g) having an average molecular weight of 200,000 g/mol is added to 100 mL of purified water and dissolved completely at 90° C., then N-acrylamide acetaldehyde (2 g) and concentrated hydrochloric acid (16 mL) are added, and the mixture is reacted at 20° C. for 6 hours. After the reaction is completed (i.e., the 6 hours passes), the pH of the reaction mixture is adjusted to 8 with aqueous sodium hydroxide. Finally, the pH-adjusted reaction mixture is concentrated until its viscosity is 2,000 cps, to obtain a sodium alginate microsphere intermediate.

S2. The above sodium alginate microsphere intermediate (10 g), 3-sulfopropyl methacrylate potassium salt (1 g), and ammonium persulfate (0.2 g) are completely dissolved in 10 mL of deionized water, then cyclohexane (63.2 mL) and Tween 20 (0.5 g) are added, and finally N,N-dimethylaniline (0.2 g) is added to the mixture under a nitrogen atmosphere and reacted at 60° C. for 4 hours. After the reaction is completed (i.e., the 4 hours passes), the reaction mixture is filtered and washed (e.g., in succession) with ethyl acetate, acetone, and deionized water to obtain crosslinked sodium alginate microspheres.

### Preparation Example 4—Preparation of Amylose Microspheres

S1. Amylose starch (15 g) having an average molecular weight of 300,000 g/mol is added to 50 g of purified water, heated to 95° C., and stirred for 3 hours. N-(2,2-dimethoxyethyl)-2-acrylamide (0.5 g) and concentrated hydrochloric acid (5 mL) are added, and the mixture is reacted at 25° C. for 5 hours. After the reaction is completed (i.e., the 5 hours passes), the pH of the reaction mixture is adjusted to 7.2 with aqueous sodium hydroxide. Finally, the pH-adjusted reaction mixture is concentrated until its viscosity is 1,800 cps, to obtain an amylose microsphere intermediate.

S2. 3-Sulfopropyl acrylate potassium salt (1.6 g) and potassium persulfate (0.86 g) are dissolved completely in 10 mL of deionized water, and the above amylose microsphere intermediate (30 g), then n-heptane (300 mL) and cellulose acetate (3.55 g) are added, and N,N-dimethylaniline (1.1 mL) is added under an inert atmosphere. This reaction mixture is reacted at 60° C. for 4 hours. After the reaction is completed (i.e., the 4 hours passes), the mixture is filtered and washed (e.g., in succession) with ethyl acetate, acetone, and deionized water to obtain crosslinked amylose microspheres.

### Preparation Example 5—Preparation of Sodium Hyaluronate Microspheres

S1. Sodium hyaluronate (20 g) having an average molecular weight of 140,000 g/mol is added to 50 g of purified water, heated to 80° C., and stirred for 2 hours. N-(2,2-dimethoxyethyl)-2-acrylamide (0.4 g) and concentrated hydrochloric acid (8 mL) are added, and the mixture is reacted at 35° C. for 3 hours. After the reaction is completed (i.e., the 3 hours passes), the pH of the reaction mixture is adjusted to 7.3 with aqueous sodium hydroxide. Finally, the pH-adjusted reaction mixture is concentrated until its viscosity is 2,000 cps, to obtain a sodium hyaluronate microsphere intermediate.

S2. The above sodium hyaluronate microsphere intermediate (20 g), sodium acrylate (1.5 g), and sodium persulfate (0.2 g) are completely dissolved in 10 mL of deionized water; then butyl acetate (180 mL) and Span 20 (1.68 g) are added, and finally triethylamine (0.32 mL) is added under an inert atmosphere. The resulting mixture is reacted at 65° C. for 6 hours. After the reaction is completed (i.e., the 6 hours passes), the reaction mixture is filtered and washed (e.g., in succession) with ethyl acetate, acetone, and deionized water to obtain crosslinked sodium hyaluronate microspheres.

### Preparation Example 6—Preparation of Sodium Carboxymethyl Cellulose Microspheres

S1. Sodium carboxymethyl cellulose (15 g) having an average molecular weight of about 90,000 g/mol is added to 50 g of purified water, heated to 90° C., and stirred for 3 hours. N-(2,2-dimethoxyethyl)-2-acrylamide (0.75 g) and concentrated hydrochloric acid (6.3 mL) were added, and the mixture was reacted at 25° C. for 5 hours. After the reaction is completed (i.e., the 5 hours passes), the pH of the reaction mixture is adjusted to 7.3 with aqueous sodium hydroxide. Finally, the pH-adjusted reaction mixture is concentrated until its viscosity is 1,500 cps, to obtain a sodium carboxymethyl cellulose microsphere intermediate.

S2. Sodium methacrylate (2.4 g) and ammonium persulfate (1.5 g) are dissolved completely in 10 mL of deionized water, and the above sodium carboxymethyl cellulose microsphere intermediate (30 g), liquid paraffin (332 mL) and Span 80 (6 g) are added, and tetramethyl ethylenediamine (1.9 mL) is added under an inert atmosphere. The mixture is reacted at 60° C. for 4 hours. After the reaction is completed (i.e., the 4 hours passes), the reaction mixture is filtered and washed (e.g., in succession) with ethyl acetate, acetone, and deionized water to obtain crosslinked sodium carboxymethyl cellulose microspheres.

### Preparation Example 7—Preparation of Glutaraldehyde-Crosslinked Polyvinyl Alcohol Microspheres

Polyvinyl alcohol (4 g) having an average molecular weight of about 80,000 g/mol is added to 40 mL of purified water at a temperature of 95° C. and stirred for 3 hours to obtain a polyvinyl alcohol solution. Part of the polyvinyl alcohol solution (10 mL) was combined with liquid paraffin (80 mL) and Span 80 (1 g) at 60° C., stirred for 2 hours, then 1 mol/L hydrochloric acid solution (2 mL) and glutaraldehyde (4 mL) are added, and the mixture reacted for 30 minutes. After the reaction is completed (i.e., the 30 minutes passes), the reaction mixture is filtered and washed three times with petroleum ether, to obtain the glutaraldehyde-crosslinked polyvinyl alcohol microspheres.

### Embodiment 1

This embodiment provides an X-ray developable embolism microsphere and a method of preparing the same. The method comprises the following steps:

### S1. Preparation of N-(2,2-dimethoxyethyl)-2,3,5-triiodobenzamide

### Synthesis Route

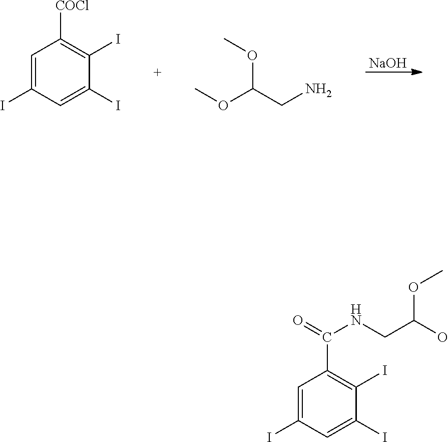

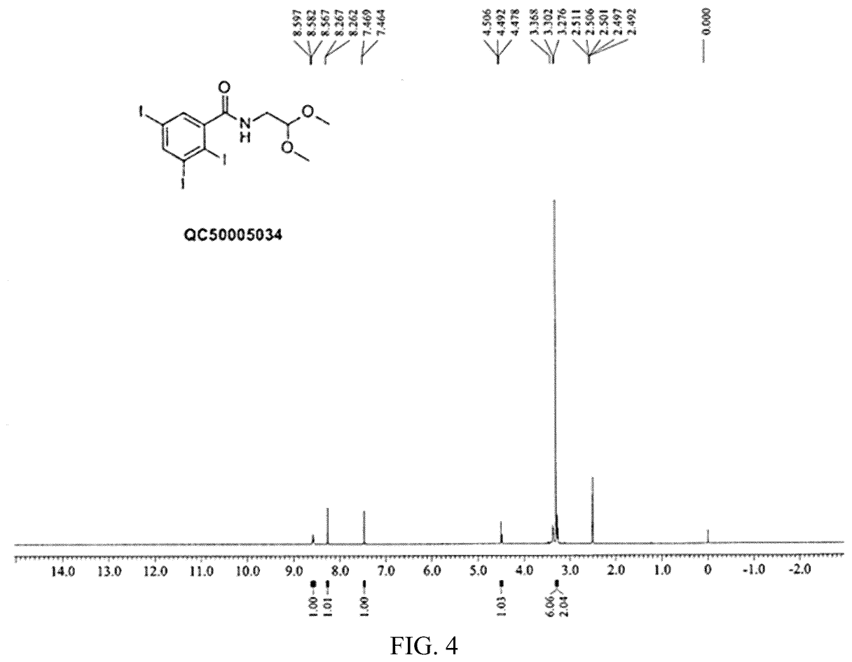

Aminoacetaldehyde dimethyl acetal (1.155 g) is dissolved in 10 mL of dimethyl sulfoxide, then 3 mol/L aqueous sodium hydroxide solution (2 mL) is added and stirred evenly. The reaction vessel is evacuated and filled with an inert gas. After cooling to −5° C., 2,4,5-triiodo-1-benzoyl chloride (5.18 g) is dissolved in dimethyl sulfoxide (50 mL) and slowly added to the reaction solution using a dropping funnel. The mixture is reacted at 30° C. for 2 hours. After the reaction is completed (i.e., the 2 hours passes), the reaction mixture is extracted twice with ethyl acetate, washed with a saturated salt solution, and then the organic phase is dried with anhydrous sodium sulfate. After filtering and removing the solvent(s) by rotary evaporation, a light yellow solid is obtained, which is N-(2,2-dimethoxyethyl)-2,3,5-triiodobenzamide. FIG. 4 shows the ¹H NMR spectroscopy of the prepared N-(2,2-dimethoxyethyl)-2,3,5-triiodobenzamide. ¹H NMR (DMSO-d₆, 400 MHz): —NH— (δ=8.58), —CH— (δ=8.26), —CH— (δ=7.46), —CH— (δ=4.49), 2-CH₃ (δ=3.30), —CH₂— (δ=3.27).

### S2. Preparation of X-Ray Developable Embolism Microspheres with N-(2,2-dimethoxyethyl)-2,3,5-triiodobenzamide

### Synthesis Route

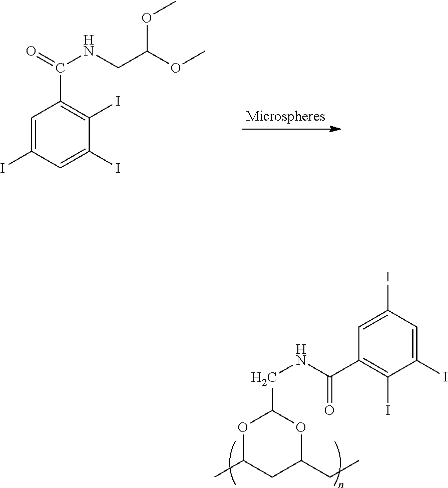

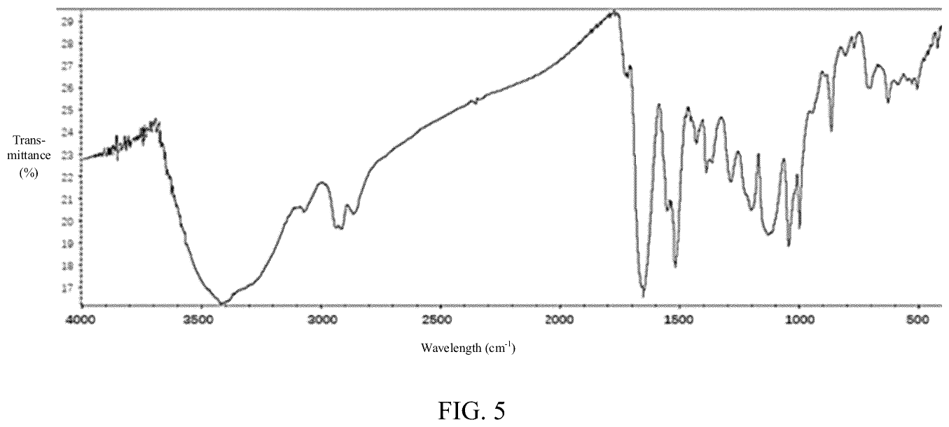

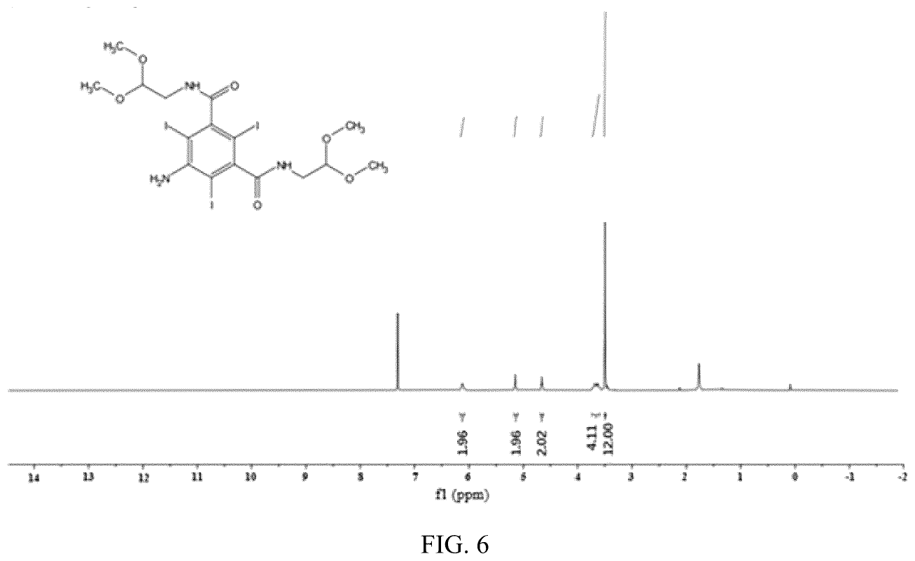

Dimethyl sulfoxide (500 mL) and the crosslinked polyvinyl alcohol microspheres prepared in Preparation Example 1 are added to a 2 L reaction flask. N-(2,2-dimethoxyethyl)-2,3,5-triiodobenzamide (prepared in S1 immediately above) is added and stirred to dissolve. Concentrated hydrochloric acid (50 mL) is slowly added (e.g., dropwise). After the acid is added, the mixture is heated to 80° C. and reacted for 2 hours. After the reaction is completed (i.e., the 2 hours passes), the reaction solvent is removed to obtain yellow particles. The particles are washed with dimethyl sulfoxide (500 mL) by stirring for 10 minutes, then filtered, washed with deionized water (500 mL) by stirring for 10 minutes, and filtered again. This washing and filtering process is repeated twice to obtain the X-ray developable polyvinyl alcohol microspheres. Yellow microspheres can be seen under a microscope. The obtained microspheres have an iodine concentration of 313 mg/g of dry microspheres. FIG. 5 shows the infrared spectrum of the prepared X-ray developable embolism microspheres, wherein peaks at 1653 cm⁻¹ and 1518 cm⁻¹ are both characteristic of amide bonds, and peaks at 869 cm⁻¹ and 706 cm⁻¹ are both characteristic of a benzene ring with substituents.

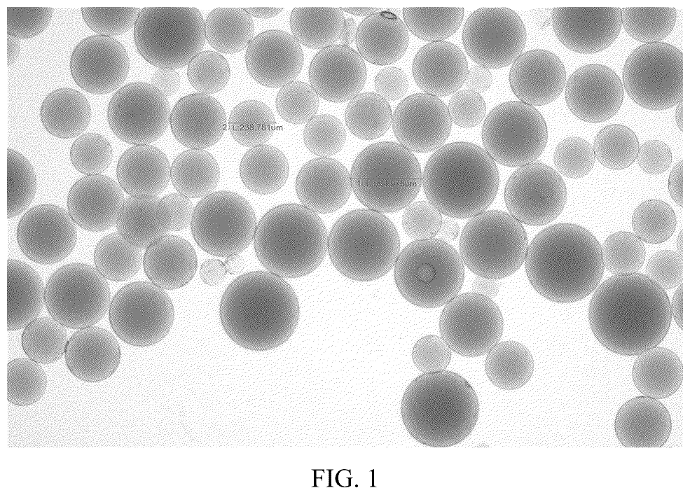

As shown in FIG. 1, the microscope image of the prepared X-ray developable embolism microspheres (magnified at 40 times) shows that, compared to the microsphere before reaction, the resulting X-ray developable microspheres change from colorless and transparent to yellow, but still maintain a good spherical shape. As shown in FIG. 1, the particle size range of the prepared microspheres is 100-500 microns.

### Embodiment 2

This embodiment provides an X-ray developable embolism microsphere and a method of preparing the same. The method comprises the following steps:

### S1. Preparation of 5-amino-1,3-bis(2,2-dimethoxyethyl)-2,4,6-triiodoisophthalamide

### Synthesis Route

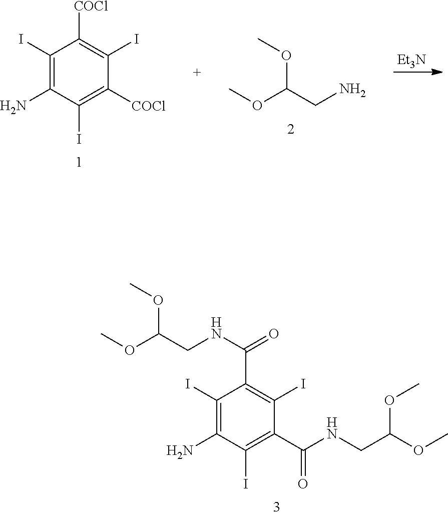

Aminoacetaldehyde dimethyl acetal (1.38 g) is dissolved in tetrahydrofuran (20 mL), then triethylamine (1.515 g) is added, and the mixture is stirred evenly. The reaction mixture is placed under an inert gas atmosphere and cooled to 0° C. 5-Amino-2,4,6-triiodoisophthaloyl dichloride (3.576 g) is dissolved in tetrahydrofuran (30 mL) and slowly added to the reaction mixture with a dropping funnel. The reaction flask is restored to the room temperature, and the reaction mixture is stirred for 24 hours. After the reaction is completed (i.e., the 24 hours passes), the reaction product is washed with deionized water, extracted twice with ethyl acetate, and finally washed with saturated salt water. After rotary evaporation to remove the solvent(s), a white or slightly off-white solid is obtained, which is 5-amino-1,3-bis(2,2-dimethoxyethyl)-2,4,6-triiodoisophthalamide. After drying, about 4.3 g of the product (compound 3) is yielded, with a product purity of greater than 99% by LC-MS. FIG. 4 shows the ¹H NMR spectroscopy of the product 5-amino-1,3-bis(2,2-dimethoxyethyl)-2,4,6-triiodoisophthalamide. ¹H NMR (CDCl₃, 400 MHz): 2 —NH— (δ=6.07), 2 —CH— (δ=5.08), —NH₂ (δ=4.69), 2 —CH— (δ=3.62), 2 —CH₃— (δ=3.56), 4 —CH₃ (δ=3.41).

### S2. Preparation of an X-Ray Developable Embolism Microsphere with 5-amino-1,3-bis(2,2-dimethoxyethyl)-2,4,6-triiodoisophthalamide

### Synthesis Route

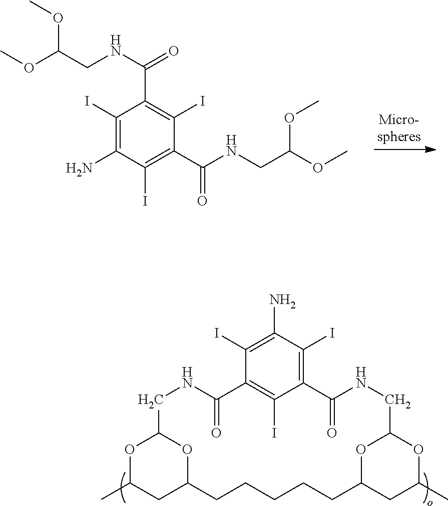

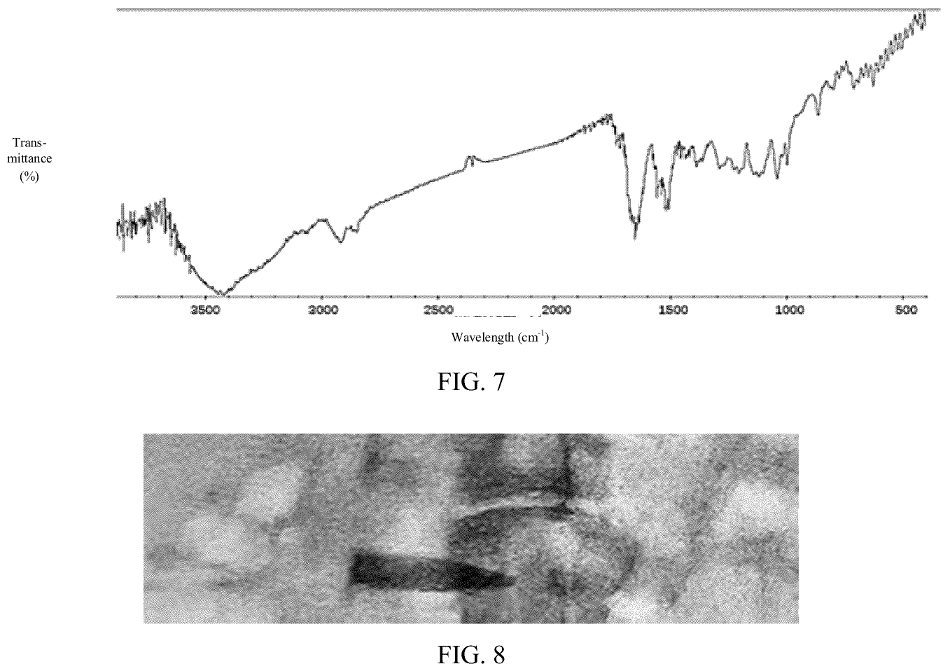

The crosslinked polyvinyl alcohol microspheres prepared in Preparation Example 2 above (4.6 g) are dispersed in deionized water (10 mL) and stirred evenly to obtain a solution of crosslinked polyvinyl alcohol microspheres. 5-Amino-1,3-bis(2,2-dimethoxyethyl)-2,4,6-triiodoisophthalamide (4 g), prepared in synthesis S1 immediately above, is dissolved in dimethyl sulfoxide (50 mL) and combined with the solution of crosslinked polyvinyl alcohol microspheres. Methanesulfonic acid (13 mL) is then added, and the mixture is stirred at room temperature for 12 hours. After the reaction is completed (i.e., the 12 hours passes), the reaction solvent(s) are removed to obtain yellow particles. The particles are washed with dimethyl sulfoxide (50 mL) (e.g., by stirring) for 10 minutes, filtered, washed with deionized water (50 mL) (e.g., by stirring) for 10 minutes, and filtered again. The washing and filtering processes are repeated twice to obtain the X-ray developable polyvinyl alcohol microspheres. Yellow microspheres can be seen under a microscope. The obtained microspheres have an iodine content of 298 mg/g of dry microspheres. FIG. 7 shows the infrared spectrum of the X-ray developable embolism microspheres prepared in this embodiment, wherein peaks at 1653 cm⁻¹ and 1520 cm⁻¹ are both characteristic of an amide bond, and peaks at 869 cm⁻¹ and 710 cm⁻¹ are both characteristic of a benzene ring with substituents.

### Embodiment 3

### S1. Preparation of N-(4-ethoxy-4-hydroxybutyl)-4-iodobenzamide

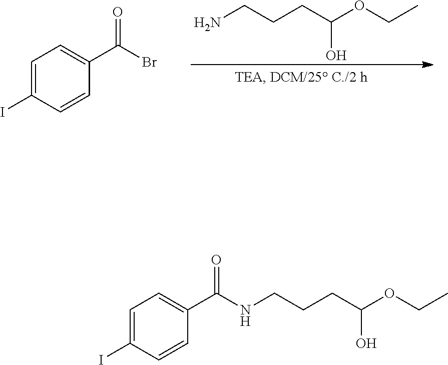

4-Iodobenzoyl bromide (3.11 g) is dissolved in dichloromethane (35 mL) in a 100 mL three-necked flask and placed under an inert gas atmosphere. The flask and its contents are cooled to 10° C., then triethylamine (5.05 g) and 4-amino-1-ethoxy-1-butanol (1.46 g) are added. The flask and its contents are restored to room temperature and reacted for 6 hours. After the reaction is completed (i.e., the 6 hours passes), the reaction mixture is added to deionized water, then extracted three times with ethyl acetate. The organic phase is washed with saturated salt solution twice, and dried with anhydrous sodium sulfate. The ethyl acetate is removed by rotary evaporation. The coarse product is thoroughly washed or pulped (isopropanol:dichloromethane=2:1) and filtered to obtain 2.85 g of N-(4-ethoxy-4-hydroxybutyl)-4-iodobenzamide, with a yield of 78%.

### S2. Preparation of an X-Ray Developable Embolism Microsphere with N-(4-ethoxy-4-hydroxybutyl)-4-iodobenzamide

### Synthesis Method

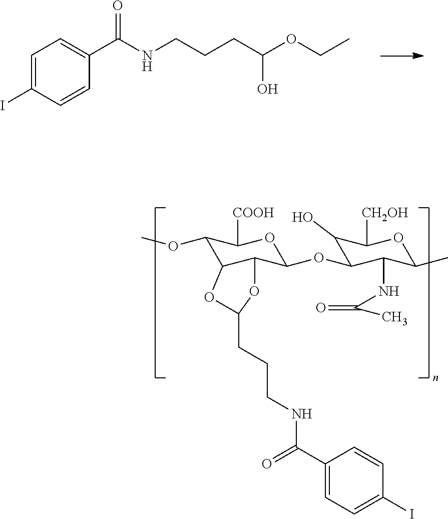

Sodium hyaluronate microspheres (1.15 g) prepared in Preparation Example 5 above are added to dimethyl sulfoxide (5 mL). N-(4-ethoxy-4-hydroxybutyl)-4-iodobenzamide (1.1 g) is dissolved in dimethyl sulfoxide (20 mL) and combined with the developing molecule solution at once. Methanesulfonic acid (1.6 mL) is added, and the reaction mixture is heated to 90° C. with stirring for 15 minutes. After the reaction is completed (i.e., the 15 minutes passes), yellow particles can be seen precipitating at the bottom of the reaction flask. The particles are washed twice with clean dimethyl sulfoxide, ethanol, and water to obtain X-ray developable sodium hyaluronate microspheres. The obtained microspheres have an iodine content of 56 mg/g of dry microspheres.

### Embodiment 4

### S1. Preparation of N-(4-formylphenyl)-2-iodobenzamide

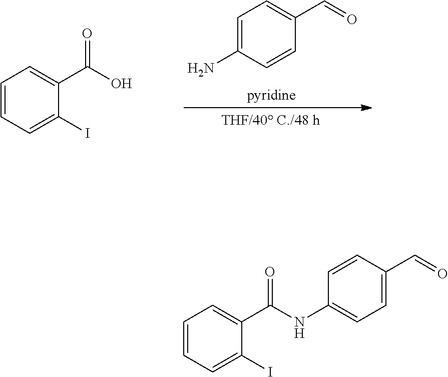 [SMILES: O=C(O)c1ccccc1I ; Nc1ccc(C=O)cc1 ; O=Cc1ccc(NC(=O)c2ccccc2I)cc1]

2-Iodobenzoic acid (2.48 g) is dissolved in tetrahydrofuran (30 mL) in a 100 mL three-necked flask, pyridine (1.58 g) is added thereto under an inert gas atmosphere, and the mixture is cooled to 5° C. A solution of tetrahydrofuran (15 mL) in which 4-aminobenzaldehyde (1.45 g) is dissolved is added to the mixture using a dropping funnel. After the addition is completed, the reaction mixture is restored to 25° C. and reacted at this temperature for 3 hours. After the reaction is completed (i.e., the 3 hours passes), the reaction solution is added to deionized water, the resulting solid is filtered, then extracted three times with ethyl acetate. The organic phase is washed with saturated salt solution twice, and dried with anhydrous sodium sulfate. The solvent is removed by rotary evaporation, purified (using ethyl acetate:n-hexane=1:9 to 1:1), and filtered using a silica gel column to obtain 1.93 g of N-(4-formylphenyl)-2-iodobenzamide after dry evaporation, with a yield of 55%.

### S2. Preparation of an X-Ray Developable Embolism Microsphere with N-(4-formylphenyl)-2-iodobenzamide

### Synthesis Method

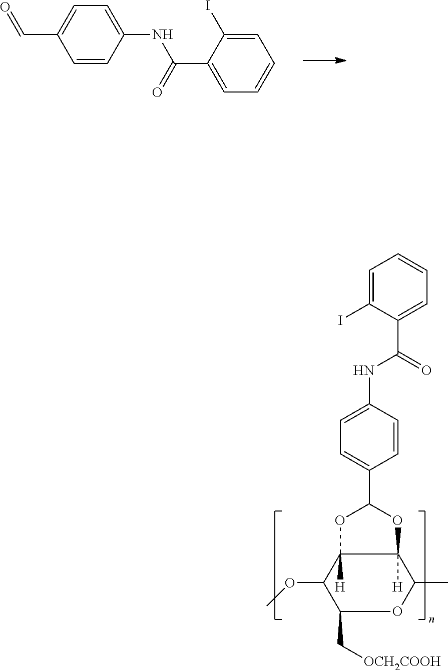

Sodium carboxymethyl cellulose microspheres (2 g) prepared in Preparation Example 6 above is added to a mixed solvent of water and acetone (10 mL), and N-(4-formylphenyl)-2-iodobenzamide (1.9 g) dissolved in acetone (40 mL) and concentrated hydrochloric acid (1 mL) are added and stirred at 60° C. for 6 hours. After the reaction is completed (i.e., the 6 hours passes), yellow particles precipitate at the bottom of the reaction flask. The particles are washed twice with clean DMSO, ethanol, and water to obtain X-ray developable sodium carboxymethyl cellulose microspheres. The obtained microspheres have an iodine content of 147 mg/g of dry microspheres.

### Embodiment 5

### S1. Preparation of 3,4-diiodo-N-(oxybutyl)benzamide

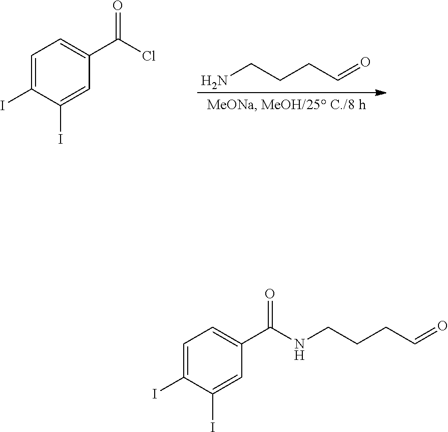 [SMILES: O=C(Cl)c1ccc(I)c(I)c1 ; NCCCC=O ; O=CCCCNC(=O)c1ccc(I)c(I)c1]

3,4-Diiodobenzoic acid (3.74 g) is dissolved in methanol (40 mL) in a 100 mL three-necked flask, cooled to 0° C., and then sodium methoxide solution (5 mol/L; 2 mL) and 4-aminobutyraldehyde are added. the flask is purged with nitrogen three times and restored to room temperature, and the reaction mixture is kept at that temperature for 8 hours. After the reaction is completed (i.e., the 8 hours passes), a citric acid solution is added to the reaction mixture, then the reaction mixture is extracted three times with ethyl acetate. The organic phase is washed with saturated salt solution twice, and dried with anhydrous sodium sulfate. The solvent(s) are removed by rotary evaporation to obtain a coarse yellow solid product. The coarse solid product is thoroughly washed or pulped (isopropyl ether), filtered, washed with isopropyl ether, and dried to obtain 3.5 g of 3,4-diiodo-N-(oxybutyl)benzamide, with a yield of 79%.

### S2. Preparation of an X-Ray Developable Embolism Microsphere with 3,4-diiodo-N-(oxybutyl)benzamide

### Synthesis Method

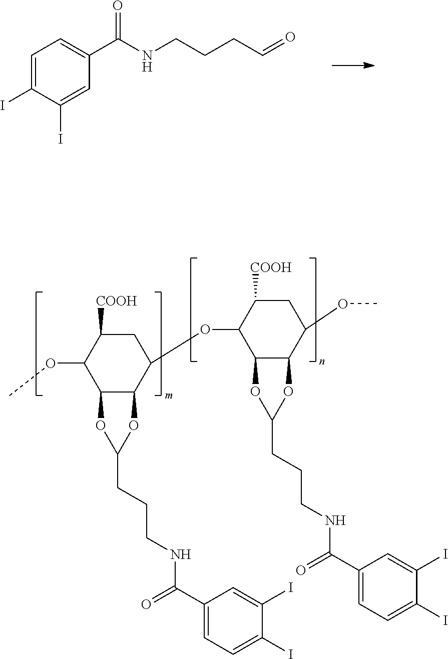

Sodium alginate microspheres (2.3 g) prepared in Preparation Example 3 above are added to acetone (50 mL) at room temperature, then 3,4-diiodo-N-(oxybutyl)benzamide (3.5 g) is added, and glacial acetic acid (8 mL) is slowly added. The mixture is stirred at 25° C. for 24 hours. After the reaction is completed (i.e., the 24 hours passes), yellow particles precipitate at the bottom of the reaction flask. The particles are washed several times with clean dimethyl sulfoxide, ethanol, and water to obtain X-ray developable sodium alginate microspheres. The obtained microspheres have an iodine content of 238 mg/g of dry microspheres.

### Embodiment 6

### S1. Preparation of 2,3,4,6-tetraiodo-N-(2-methyl-3-acraldehyde)benzamide

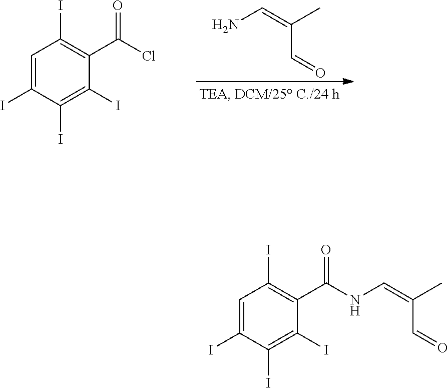

2,3,4,6-Tetraiodobenzoyl chloride (2.48 g) and 3-amino-2-methylpropenal (0.85 g) are dissolved in dichloromethane (35 mL) in a 50 mL three-necked flask at 0° C., then triethylamine (2.92 g) is added, and the flask is purged with nitrogen three times. The reaction mixture is stirred at 0° C. for 48 hours. Alternatively, the reaction mixture may be stirred at room temperature for 24 hours. After the reaction is completed (i.e., the 24 or 48 hours passes), the reaction mixture is added to water, then extracted three times with ethyl acetate. The organic phase is washed with saturated salt solution twice, and dried with anhydrous sodium sulfate. The solvent(s) are removed by rotary evaporation. The coarse product is purified by passing through a silica gel column (ethyl acetate:n-hexane=1:9 to 7:3) to obtain 2,3,4,6-tetraiodo-N-(2-methyl-3-acraldehyde)benzamide (3.6 g) after rotary evaporation, with a yield of 52%.

### S2. Preparation of an X-Ray Developable Embolism Microsphere with 2,3,4,6-tetraiodo-N-(2-methyl-3-acraldehyde)benzamide

### Synthesis Method

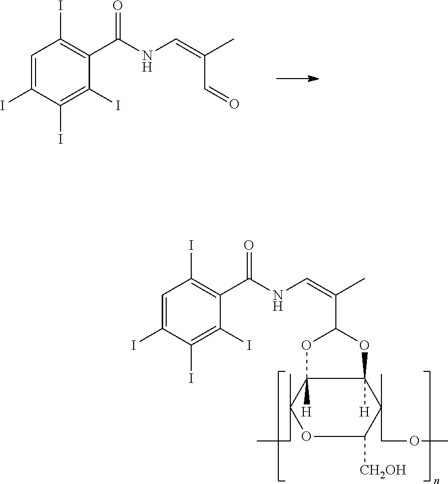

The amylose microspheres (4.6 g) prepared in Preparation Example 4 above are added to acetonitrile (100 mL) at room temperature, then 2,3,4,6-tetraiodo-N-(2-methyl-3-acraldehyde)benzamide (3.1 g) is added and cooled to 0° C. Perchloric acid (0.5 mL) is slowly added, and the mixture is stirred at 75° C. for 1 hour. After the reaction is completed (i.e., the 1 hour passes), yellow particles can be observed precipitating at the bottom of the reaction flask. The particles are washed several times with clean DMSO, ethanol, and water to obtain X-ray developable amylose microspheres. The obtained microspheres have an iodine content of 179 mg/g of dry microspheres.

### Embodiment 7

### S1. Preparation of N-(2,2-dimethoxyethyl)-2,3,5-triiodobenzamide

Same as S1 in Embodiment 1.

### S2. Preparation of an X-Ray Developable Embolism Microsphere with N-(2,2-dimethoxyethyl)-2,3,5-triiodobenzamide

Dimethyl sulfoxide (100 mL) and the glutaraldehyde-crosslinked polyvinyl alcohol microspheres (10 g) prepared in Preparation Example 7 above are added to a 2 L reaction flask. N-(2,2-dimethoxyethyl)-2,3,5-triiodobenzamide (3 g), prepared in synthesis S1 immediately above, are added to the flask and dissolved by stirring. Concentrated hydrochloric acid (10 mL) is slowly added (e.g., dropwise). Thereafter, the reaction mixture is heated to 80° C. and reacted for 1 hour. After the reaction is completed (i.e., the 1 hour passes), the solvent(s) are removed to obtain yellow particles. The particles are then washed with dimethyl sulfoxide (100 mL) by stirring for 10 minutes, then the solvent is removed (e.g., by filtering). The particles are then washed with deionized water (100 mL) by stirring for 10 minutes and filtered. The washing and filtering processes are repeated twice to obtain the X-ray developable glutaraldehyde-crosslinked polyvinyl alcohol embolism microspheres. The obtained microspheres have an iodine concentration of 102 mg/g of dry microspheres.

### Embodiment 8: Testing the X-Ray Developing Properties of the Microspheres

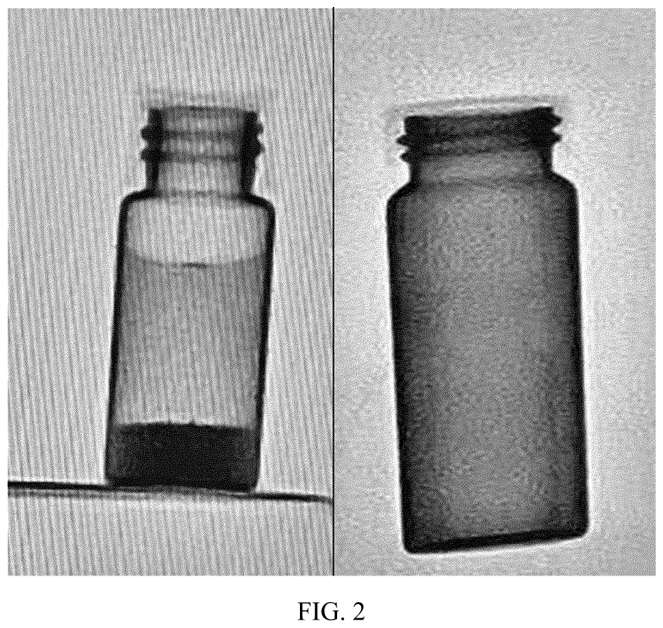

The X-ray developable polyvinyl alcohol embolism microsphere obtained from Embodiment 1 is soaked in normal saline and placed in a penicillin bottle to observe the developing properties of the microspheres using digital subtraction angiography (DSA) technology (voltage 64 kV, current 160 mA, distance 100 cm). FIG. 2 shows an X-ray image of the X-ray developable polyvinyl alcohol embolism microsphere (left) and of non-developable polyvinyl alcohol embolism microspheres (right) using digital subtraction angiography (DSA) technology. According to FIG. 2, the present microspheres show an obvious contrast in X-ray imaging.

The X-ray developable glutaraldehyde-crosslinked polyvinyl alcohol embolism microspheres obtained in Embodiment 7 are soaked in normal saline and placed in a 1 mL centrifuge tube, then photographed using X-rays. As shown in FIG. 8, the present microspheres prepared show an obvious contrast in X-ray imaging.

### Embodiment 9: Testing the Drug Loading Properties of the X-Ray Developable Embolism Microspheres

Surface moisture on the X-ray developable embolism microspheres prepared in Embodiment 1, Embodiment 2, Embodiment 3, Embodiment 4, Embodiment 5, Embodiment 6 and Embodiment 7 was removed, 1 g of the microspheres was weighed into a penicillin bottle, 4 mL of 20 mg/mL aqueous doxorubicin hydrochloride solution was added, and the penicillin bottle was sealed and placed on a flat plate oscillator to oscillate at 180 rpm. 10 μl of the sample was removed at pre-set time points and diluted to 2 mL. The concentration of doxorubicin hydrochloride solution is tested at 480 nm using a UV spectrophotometer to calculate the drug adsorption/absorption capacity and drug loading rate of the embolism microspheres. The drug loading rate data are shown in Table 1.

TABLE 1
Drug Loading Rate Testing Data of X-ray Developable Embolism Microspheres

| Adsorption | Drug loading rate | | | | | | |
|---|---|---|---|---|---|---|---|
| time (min) | Embodiment 1 | Embodiment 2 | Embodiment 3 | Embodiment 4 | Embodiment 5 | Embodiment 6 | Embodiment 7 |
| 0 | 0.00% | 0.00% | 0.00% | 0.00% | 0.00% | 0.00% | 0.00% |
| 15 | 86.60% | 86.17% | 79.82% | 76.59% | 81.78% | 75.08% | 25.11% |
| 30 | 90.28% | 90.18% | 79.27% | 77.77% | 83.46% | 77.41% | 29.56% |
| 45 | 93.00% | 93.35% | 82.07% | 81.95% | 85.18% | 83.17% | 33.06% |
| 60 | 94.62% | 94.80% | 86.41% | 84.99% | 86.81% | 88.11% | 35.02% |
| 120 | 95.82% | 96.07% | 89.38% | 87.91% | 88.76% | 90.54% | 37.12% |
| 180 | 97.57% | 96.80% | 92.48% | 91.36% | 90.97% | 94.19% | 38.26% |
| 240 | 98.77% | 97.90% | 93.58% | 91.74% | 92.77% | 95.41% | 38.61% |

### Comparative Example 1

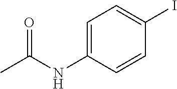

### Preparation of Embolism Microspheres with N-(4-Iodophenyl)Acetamide

### Synthesis Method

Polyvinyl alcohol microspheres (2 g) are dispersed in dimethyl sulfoxide (5 mL), then N-(4-iodophenyl)acetamide (1.6 g) is dissolved in dimethyl sulfoxide (15 mL) and the microsphere dispersion is added thereto. Concentrated hydrochloric acid (2 mL) is added, and the mixture is stirred at 80° C. for 2 hours. After the reaction is completed (i.e., the 2 hours passes), transparent beads can be observed at the bottom of the reaction flask. The beads are washed several times with clean dimethyl sulfoxide, ethanol, and water, then boiled in a phosphate-buffered saline solution and restored to room temperature for storage and observation of the resulting microspheres. The color of the microspheres does not change significantly compared to that before the reaction.

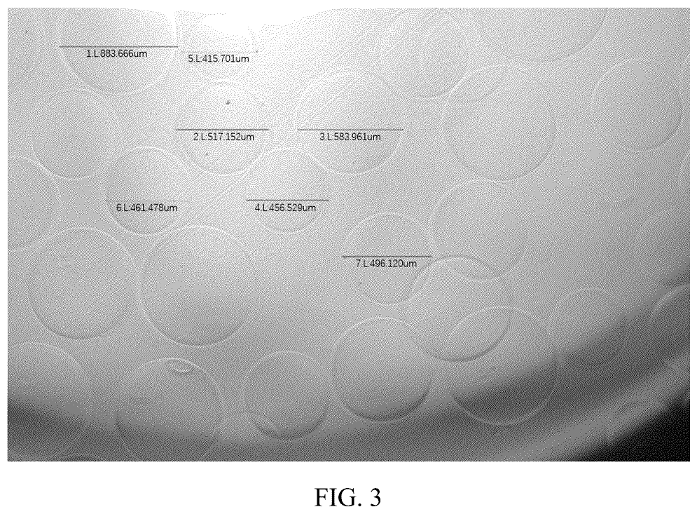

FIG. 3 is a micrograph of the microspheres prepared in this Comparative Example. From FIG. 3, it can be seen that the microspheres remain transparent after the reaction and have no X-ray developing effect, indicating that N-(4-iodophenyl)acetamide is not connected to the microspheres. This is because N-(4-iodophenyl)acetamide does not have a functional group that can react with the polyhydroxy polymer microspheres.

### Comparative Example 2

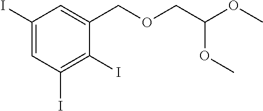 [SMILES: COC(COCc1cc(I)cc(I)c1I)OC]

### Preparation of X-Ray Developable Embolism Microspheres with 1-(2,2-dimethoxyethoxymethyl)-2,3,5-triiodobenzene

### S1. Synthesis of 1-(2,2-dimethoxyethoxymethyl)-2,3,5-triiodobenzene

2,3,5-triiodobenzyl alcohol (5.07 g) is dissolved in anhydrous 2-methyltetrahydrofuran (55 mL) under a nitrogen atmosphere, then 2-bromo-1,1-dimethoxyethane (2.11 g) and sodium hydride (0.54 g) are added. The reaction mixture is heated to reflux for 17 hours under the nitrogen atmosphere. After the reaction is completed (i.e., the 17 hours passes), the reaction mixture is dissolved in dichloromethane (50 mL) and washed four times with deionized water (25 mL). The organic layer is concentrated under vacuum to obtain 1-(2,2-dimethoxyethoxymethyl)-2,3,5-triiodobenzene.

### S2. Preparation of an X-Ray Developable Embolism Microsphere

The process of Embodiment 1 is followed, except that N-(2,2-dimethoxyethyl)-2,3,5-triiodobenzamide is replaced with 1-(2,2-dimethoxyethoxymethyl)-2,3,5-triiodobenzene having a similar molar mass, and the other steps are the same as those in Embodiment 1. The obtained microspheres have an iodine content of 20 mg/g of dry microspheres.

Through comparison, the synthesis method of this compound is more complex, with a longer reaction time and more intense reaction conditions, and uses sodium hydride, a relatively dangerous reagent. In addition, compared to the present X-ray developing compounds with amide bonds, the coupling yield between 1-(2,2-dimethoxyethoxymethyl)-2,3,5-triiodobenzene and the microspheres is relatively low. This may be due to the relatively high polarity of the amide bond, which results in better solubility in polar solvents. In addition, compounds with amide bonds generally have better affinity with the polymer network of the microspheres, and are more stable when the amide compounds are connected to the polymer chain, resulting in a higher degree of reaction.

The foregoing are only selected embodiments of the present invention, and are not intended to limit the present invention. Any modifications, equivalents, replacements, improvements, etc. made within the spirit and principles of the present invention shall be covered within the scope of protection for the present invention.

What is claimed is:

1. An X-ray developable molecule, having the Formula I: 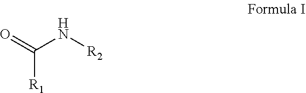
wherein R₁ is a phenyl group substituted with 1 to 4 iodine atoms, 0 or 1 —NH₂ groups, and 0 or 1 —C(═O)—NH—R₂ groups, and R₂ is a structure containing an aldehyde, a hemiacetal, or an acetal.

2. The X-ray developable molecule according to claim 1, wherein R₁ is selected from the group consisting of:

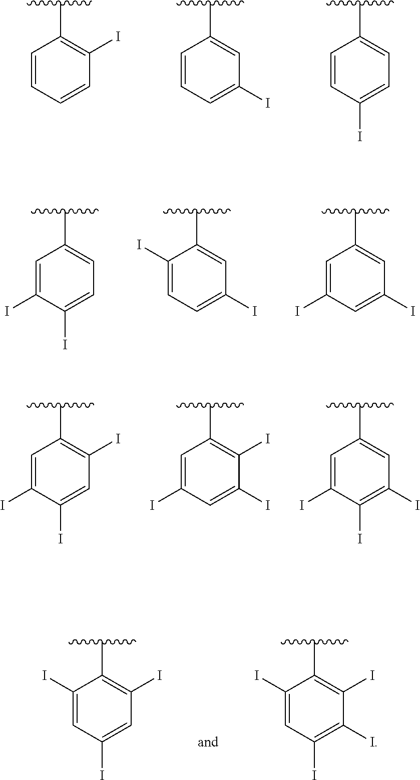

3. The X-ray developable molecule according to claim 1, wherein R₂ is selected from the group consisting of: 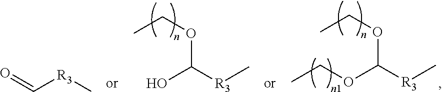
where R₃ is an aryl group or a saturated or unsaturated C₁-C₆ alkylene group, and n and n₁=0-3.

4. The X-ray developable molecule according to claim 1, comprising a compound selected from the group consisting of:

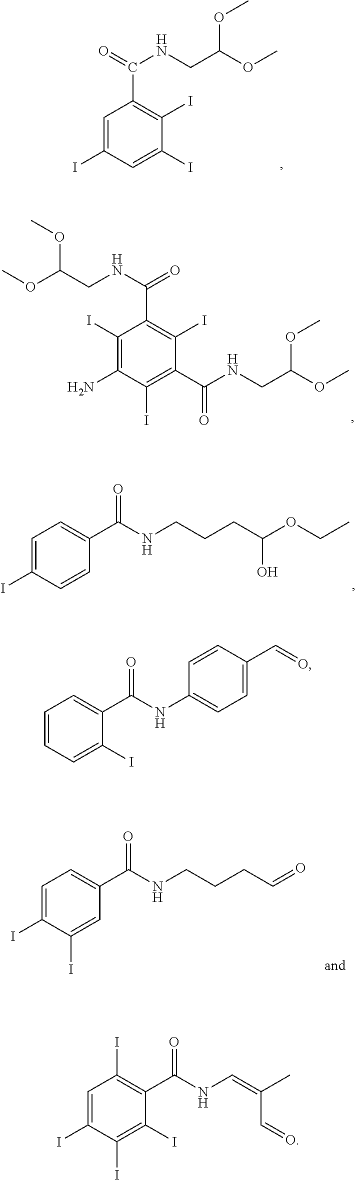

5. A method of preparing the X-ray developable molecule according to claim 1, comprising reacting a compound containing an amino group and the aldehyde, hemiacetal or acetal group with an iodobenzoic acid derivative.

6. The method according to claim 5, wherein the compound is: 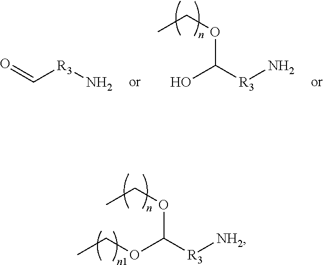
where R₃ is an aryl group or a saturated or unsaturated C₁-C₆ alkylene group, and n and n₁=0-3.

7. The method according to claim 5, wherein the iodobenzoic acid derivative contains both the R₁ group and a hydroxyl, a carboxyl, an acyl chloride or an acyl bromide group.

8. The method according to claim 5, wherein reacting the compound with the iodobenzoic acid derivative comprises adding the compound, the iodobenzoic acid derivative and an alkali to an organic solvent, reacting the compound, the iodobenzoic acid derivative and the alkali at a temperature of 0-40° C. for a time of 0.5-48 hours, and removing the organic solvent to obtain the X-ray developable molecule.

9. The method according to claim 8, wherein the alkali is selected from the group consisting of sodium hydroxide, potassium hydroxide, diethylamine, ethylenediamine, triethylamine, ammonia, pyridine, sodium methoxide, and sodium hydride; and the organic solvent is selected from the group consisting of dimethyl sulfoxide, tetrahydrofuran, dichloromethane, chloroform, methanol, acetone, acetonitrile, diethyl ether, N-methylpyrrolidone, and N,N-dimethylformamide.

10. An X-ray developable embolism microsphere, comprising a polyhydroxy polymer microsphere and the X-ray developable molecule of claim 1, bound or coupled to the polyhydroxy polymer microsphere through an acetal group or link.

11. The X-ray developable embolism microsphere according to claim 10, wherein the polyhydroxy polymer microsphere comprises a crosslinked copolymer of a polyhydroxy polymer with a water-soluble molecule containing one or more unsaturated bonds and one or more aldehyde or acetal groups.

12. The X-ray developable embolism microsphere according to claim 11, wherein the copolymer is cross-linked by a crosslinking agent selected from the group consisting of carboxylic acid compounds and carboxylate salts thereof having or generating one or more carboxylate groups and having one or more unsaturated bonds, and sulfonate compounds and sulfonic acid compounds having or generating one or more sulfonate groups and having one or more unsaturated bonds.

13. The X-ray developable embolism microsphere according to claim 10, wherein the polyhydroxy polymer microsphere comprises a polyhydroxy polymer crosslinked with a water-miscible compound containing two or more aldehyde or acetal groups.

14. The X-ray developable embolism microsphere according to claim 10, wherein the X-ray developable embolism microsphere contains an amount of iodine greater than or equal to 30 mg/g of the microsphere.

15. A method of preparing the X-ray developable embolism microsphere according to claim 10, comprising dissolving the polyhydroxy polymer microsphere and the X-ray developable molecule in a solvent, adding an acid, reacting the polyhydroxy polymer microsphere and the X-ray developable molecule, and removing the solvent.

16. The method according to claim 15, wherein the polyhydroxy polymer microsphere comprises a polymer or polysaccharide selected from the group consisting of polyvinyl alcohol, chitosan, hyaluronates, alginates, amylose, cellulose and modified cellulose.

17. The method according to claim 15, wherein the acid is selected from the group consisting of hydrochloric acid, sulfuric acid, nitric acid, methanesulfonic acid, acetic acid, citric acid, benzoic acid, and perchloric acid.

18. The method according to claim 15, wherein the solvent is selected from the group consisting of dimethyl sulfoxide, water, acetone, acetonitrile, and N-methylpyrrolidone.

19. The method according to claim 15, further comprising:
dissolving a polyhydroxy polymer in water, then adding a water-soluble molecule containing one or more unsaturated bonds and one or more aldehyde or acetal groups, and an inorganic acid;
reacting the polyhydroxy polymer with the water-soluble molecule to form a solution containing a microsphere intermediate;
after reacting the polyhydroxy polymer with the water-soluble molecule, adjusting the pH to 7-9, concentrating the pH-adjusted solution, and obtaining the microsphere intermediate;
dissolving the microsphere intermediate, a crosslinking agent containing one or more anionic functional groups and one or more unsaturated bonds, and an initiator in water;
adding a solvent, a surfactant, and an organic alkali to the water;
reacting the microsphere intermediate, the crosslinking agent, and the initiator under an inert gas atmosphere, and
after reacting the microsphere intermediate, the crosslinking agent, and the initiator, filtering and washing a resulting reaction mixture to obtain the polyhydroxy polymer microsphere.

20. The method according to claim 19, wherein the polyhydroxy polymer with the water-soluble molecule are reacted at a temperature of 10-35° C. for a length of time of 3-8 hours; and the microsphere intermediate, the crosslinking agent, and the initiator are reacted at a temperature of 55-65° C. for a length of time of 2-6 hours.
